(19) United States
(12) Patent Application Publication
(10) Pub. No.: US 2008/0255639 A1
(43) Pub. Date: Oct. 16, 2008

(54) METHOD AND DEVICE FOR TREATING TISSUE USING A COAGULATED BEAM PATH

(75) Inventors: Oliver Stumpp, Palo Alto, CA (US); Christin T. Piazza, Los Gatos, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 12/102,740

(22) Filed: Apr. 14, 2008

Related U.S. Application Data
(60) Provisional application No. 60/911,854, filed on Apr. 13, 2007.

(51) Int. Cl. A61B 18/20

(52) U.S. Cl. 607/89

(57) ABSTRACT

The invention disclosed herein is directed to methods and devices for treating tissue having an overlying portion and an underlying portion by using a first fractional optical energy treatment to coagulate a plurality of zones in the overlying portion, thereby reducing the optical scattering of the overlying portion, and directing a subsequent fractional optical energy treatment through the coagulated zones to the underlying portion in order to produce an effective treatment in the underlying portion of a region of tissue. The methods and devices disclosed can further comprise detection and treatment of subsurface targets in the underlying portion of tissue. The treatment parameters used to deliver the first and subsequent treatments, including wavelength, can be optimized in order to provide a first optical treatment that effectively coagulates the overlying portion and a subsequent one or more optical treatments that effectively treat the underlying portion.

### CROSS-REFERENCE TO RELATED APPLICATIONS

This application claims the benefit of and priority under 35 U.S.C. §119(e) to U.S. Provisional Patent Application Ser. No. 60/911,854, “Method and Device for Treating Tissue Using a Coagulated Beam Path,” by inventors Oliver Stumpp and Christin T. Piazza, filed Apr. 13, 2007, which is incorporated herein by reference in its entirety.

### FIELD OF THE INVENTION

This invention relates generally to methods and devices for using optical energy to treat tissue having an overlying portion and an underlying portion so as to provide an effective treatment of the underlying portion of tissue. More particularly, it relates to methods and devices to treat tissue using optical energy in a fractional manner by first forming a coagulated zone having reduced optical scattering in an overlying portion of a region of tissue, and subsequently treating an underlying portion by directing optical energy through the coagulated zone to the underlying portion in a manner so as to effectively treat a condition present in the underlying portion of the region of tissue.

### INTRODUCTION

Methods and devices capable of treating subsurface targets such as, for example, the hair bulb or hair bulge in the dermis for hair removal, or vascular lesions in the dermis, or laser tattoo removal, are useful for a large population of patients. Many optical energy based treatments have been developed to treat subsurface targets, but not all have been able to penetrate as deeply as needed so as to provide effective treatments of the subsurface targets without causing unwanted side effects. One reason for this is that the optical energy is absorbed and scattered by the overlying portion of tissue, such as, for example, the epidermis of the skin, making it difficult to deliver adequate levels of optical energy to the underlying portion of tissue, such as, for example, the dermis or subcutis.

One method which has been used to treat subsurface targets is to use bulk treatments which provide high fluences over an entire portion of tissue so that an adequate amount of the treatment energy gets through the overlying portion of tissue and reaches the underlying portion of tissue. These treatment approaches, however, can cause adverse effects in the overlying portion of tissue, such as, for example, scarring and pigment changes in the epidermis. Cooling the overlying portion of tissue can reduce damage and the adverse effects of the treatment on the overlying portion, but the necessity for cooling significantly complicates the apparatus required to provide the treatment and is not as effective as desired.

Another method which has been used to solve this problem is the use of topically applied optical skin clearing agents, such as glycerol, polyethylene glycol, dextrose solutions, and the like. While the use of these hyperosmotic agents can reduce optical scattering in highly turbid skin, it can take a considerable amount of time for these agents to produce optical clearing, and their efficacy is highly dependent upon the technique used to apply the agents.

Clearly, a need remains for methods and devices capable of delivering optical energy treatments that can provide effective treatments to underlying portions of tissue without producing undesired effects in the overlying portions of tissue, and which can be rapidly and consistently delivered.

The invention described herein uses fractional optical energy based treatment methods to reduce the optical scattering in an overlying portion so as to allow more optical energy to penetrate the overlying portion in order to reach an underlying portion, to effectively treat the underlying portion.

### SUMMARY OF THE INVENTION

The invention disclosed herein is directed to methods and devices for treating a region of tissue having an overlying portion and an underlying portion by using a first optical energy treatment to produce zones of coagulated tissue in the overlying portion, and using the coagulated zones in the overlying portion as beam paths in order to direct a subsequent optical energy treatment through the coagulated zones to the underlying portion, in order to produce an effective treatment in the underlying portion of a region of tissue. These methods and devices allow effective optical energy based treatments to be rapidly and consistently provided in an efficient manner to the underlying portion of a region of tissue.

In one example, the method comprises treating an overlying portion of tissue with a first optical energy treatment in a fractional manner so as to thermally coagulate a plurality of fractions of the overlying portion of a region of tissue, thereby creating a plurality of coagulated zones having reduced light scattering as compared to equivalent sized zones of untreated overlying tissue; and treating an underlying portion of tissue with a subsequent optical energy treatment in a fractional manner so as to effectively treat a condition present in the underlying portion of tissue, thereby creating at least one treatment zone in the underlying portion of tissue, wherein the subsequent optical energy treatment is directed through at least one of the plurality of coagulated zones in the overlying portion to form the at least one treatment zone in the underlying portion of a region of tissue. In another example, the method can further comprise detecting a subsurface target in the region of tissue. In another example, the method can further comprise detecting a subsurface target in the region of tissue through the coagulated zone. In yet another example, the method comprises delivering the subsequent optical energy treatment to a detected subsurface target.

In another example, a device for providing an optical energy treatment to a region of tissue having an overlying portion and an underlying portion is disclosed. The device comprises an optical energy source for providing a first optical energy treatment configured to apply the first optical energy treatment in a fractional manner so as to thermally coagulate a plurality of fractions of an overlying portion of a region tissue, thereby creating a plurality of coagulated zones having reduced light scattering as compared to equivalent sized zones of untreated overlying tissue; an optical energy source for providing a subsequent optical energy treatment configured to apply the subsequent optical energy treatment in a fractional manner so as to effectively treat a condition present in the underlying portion of tissue, thereby creating at least one treatment zone in the underlying portion of tissue, wherein the subsequent optical energy treatment is directed through at least one of the plurality of coagulated zones in the overlying layer in order to form the at least one treatment zone in the underlying portion of a region of tissue; a controller configured to control the optical energy source or sources providing the first and subsequent optical energy treatments; and a detector configured to detect the presence of a subsurface target through at least one of the plurality of coagulated zones and to provide feedback to the controller; wherein the controller uses the feedback from the detector to determine whether or not to apply the subsequent optical energy treatment to a detected subsurface target through the at least one of the plurality of coagulated zones in order to effectively treat the detected subsurface target by creating the at least one treatment zone in the underlying portion. In one example, the spot size of the first optical energy treatment is larger than the spot size of the subsequent optical energy treatment. In another example, the spot size of the first optical energy treatment is smaller than the spot size of the subsequent optical energy treatment. In yet another example, the spot size of the first and subsequent optical energy treatments are approximately equal.

The methods of treatment and devices described herein can be used to treat a number of subsurface targets such as, for example, the hair bulb or hair bulge in the dermis, vascular lesions in the dermis, pigmented lesions in the dermis, subcutaneous fat deposits, cellulite, tattoo ink, etc. Similarly, the methods of treatment and devices described herein can be used for a number of indications, such as, for example, hair removal, removal of vascular lesions, removal of pigmented lesions, removal of tattoos, reduction of subcutaneous fat, reduction of cellulite, etc.

Other aspects of the invention include methods corresponding to the devices described herein.

### DETAILED DESCRIPTION

Fractional treatment methods involve the generation of a large number of treatment zones within a region of tissue. In fractional optical energy based treatments, the optical energy impacts directly on only a number of relatively small zones, instead of impacting directly on a larger region of tissue undergoing treatment, as it does in conventional bulk treatments. Thus, a region of skin treated using optical energy delivered in a fractional manner is composed of a plurality of zones where the tissue has been altered by the optical energy, where the plurality of zones are contained within a larger volume of tissue that has not been altered by the optical energy. Fractional treatment methods make it possible to leave substantial volumes of tissue unaltered and viable within a region of tissue undergoing treatment.

Fractional treatment methods have been used to provide effective treatments for both treatment of existing medical (e.g., dermatological) disease conditions and for treatment aimed at improving the appearance of tissue (e.g., skin) by intentionally generating zones of thermally altered tissue amongst zones of unaltered tissue and/or lesser-treated tissue. Fractional treatment methods offer numerous advantages over existing approaches in terms of safety and efficacy. Fractional treatment methods minimize the undesirable side effects of pain, erythema, swelling, fluid loss, prolonged reepithelialization, infection, and blistering generally associated with bulk optical energy based treatments of tissue. By sparing healthy tissue around the zones of thermally altered tissue, fractional treatment methods increase the rate of recovery of the altered zones by stimulating remodeling and wound repair mechanisms. Fractional treatment methods also reduce or eliminate the side effects of repeated optical energy treatments to tissue by controlling the extent of tissue necrosis due to exposure to optical energy.

Treating tissue with optical energy can produce many different types of effects in the tissue, including denaturation, coagulation, cell necrosis, melting, welding, retraction, alteration of the extra-cellular matrix, charring, and ablation. The type of effect or effects produced in the tissue, the depth to which the effect or effects extend into in the tissue, as well as the diameter of the zone of tissue effected by the optical energy, are dependent upon the treatment parameters used. These treatment parameters include the wavelength, the total irradiance, the local irradiance, the total fluence, the local fluence, the pulse energy, the pulse duration, the pulse repetition rate, the spot size of the treatment beam, the density of zones treated per square centimeter of tissue surface for fractional treatments, etc. The condition of the tissue (e.g., the hydration level of the tissue, the level of chromophores present in the tissue, etc.) can also affect the type of effect or effects produced in the tissue, the depth to which the effect or effects extend into the tissue, and the diameter of the zone of tissue affected by the optical energy.

Treatment of tissue with optical energy in a manner so as to cause thermal coagulation of the tissue, while causing necrosis of the coagulated zone, produces a thermal wound that can be rapidly repaired by the surrounding living tissue and, under many conditions, does not result in adverse effects, such as, for example, scarring or pigmentation changes in skin. Producing coagulated zones of tissue using fractional treatment methods can further reduce the incidence of adverse effects. Methods of using fractional photothermolysis to create microscopic lesions that allow for dermal content to be exfoliated through the stratum corneum are described, for example, in U.S. patent application Ser. No. 11/548,248, which is herein incorporated by reference.

Treatment of tissue with optical energy in a manner so as to cause thermal coagulation of the tissue also reduces the optical scattering properties of the tissue, producing a thermally-induced “optical clearing” of the tissue. Treating tissue with optical energy in a fractional manner so as to cause thermal coagulation can produce a number of coagulated zones with reduced optical scattering. The reduced optical scattering of these coagulated zones allow them to be used as “beam paths” by aiming optical energy through the coagulated zones to a deeper portion of the tissue beneath the coagulated zone. Using the coagulated zones as beam paths allows a greater amount of optical energy to reach underlying portions of tissue, as less optical energy is lost to scattering through the coagulated zones than would be lost through untreated zones.

In some cases, the level and type of treatment that effectively coagulates the overlying portion of tissue can also be effective to treat the underlying portion. However, in other cases, treatment of the underlying portion of tissue can require more or less optical energy, or may require the use of a different wavelength of optical energy in order to be effectively treated. In these cases, the coagulated zones created by the first optical energy treatment can be used as beam paths to deliver a subsequent optical energy treatment to effectively treat the underlying portion of tissue. This allows the subsequent optical energy treatment to be optimized to effectively treat a condition present in an underlying portion of tissue, or to be optimized for delivery directly to the underlying portion through the beam path. When used alone, a subsequent optical energy treatment that is effective to treat the underlying portion when directed through a coagulated zone may not be powerful enough to penetrate an untreated overlying portion, or may be too highly scattered by the untreated overlying portion to reach the underlying portion. Alternatively, when used alone, a subsequent optical energy treatment that is effective to treat the underlying portion when directed through a coagulated zone may be so powerful that it would cause adverse effects if it were to be applied directly to an untreated overlying portion of the tissue. By using the method of applying a first optical energy treatment to create coagulated zones which act as beam paths, subsequent optical energy treatments that either cannot not reach the underlying portion or that can damage an untreated overlying layer can be applied through the beam paths directly to the underlying portion, allowing the underlying portion to be treated using an optimal subsequent optical energy treatment.

For some treatments, it can be desirable to provide a first treatment that produces ablation in addition to coagulation in the treatment zones. For example, the first treatment can be a combination of an ablative treatment which removes all or or a portion of the stratum corneum and/or epidermis and a coagulative treatment which forms coagulation zones below the ablated region to act as beam paths for one or more subsequent optical energy treatments. Removal of all or a portion of the stratum corneum and/or epidermis and the formation of a coagulated beam path below the ablated region can allow the subsequent treatment(s) to more effectively reach target locations in the dermis and/or subcutaneous tissue.

The first and subsequent optical energy treatments can be delivered with the first treatment immediately followed by one or more subsequent treatments, with the first treatment partially overlapping in time with a subsequent treatment, or with a gap in time between the first treatment and the one or more subsequent treatments. The duration of the gap in time between the first treatment and the one or more subsequent treatments is limited by the amount of time before the coagulation zone heals and is replaced by normal, untreated tissue.

Similarly, the number of optical energy treatments that can be subsequently applied to the underlying portion of the tissue is limited by the amount of time before the coagulation zone heals and is replaced by normal, untreated tissue. As the coagulated tissue in a beam path has already been necrosed by the initial treatment, subsequent optical energy treatments using very high fluences can be applied to the coagulated zones without producing significant adverse effect on the surrounding living tissue in the overlying layer. In one example, the number and intensity of subsequent optical energy treatments is limited by the amount of optical energy required to char or ablate the coagulated zones. Subsequent treatments that result in substantial charring of the coagulation zone can be undesirable in many indications as substantial charring can lead to adverse effects in the overlying portion. As the aim of the first optical energy treatment is to produce a zone of coagulated tissue with reduced light scattering, first optical energy treatments that result in substantial charring of the overlying portion of tissue are undesirable as they do not produce zones of decreased light scattering, and are to be avoided.

Depending upon the optical properties of an untreated underlying portion of tissue, optical energy that passes through a coagulated zone in an overlying portion to an untreated underlying portion can scatter in an increased fashion when it encounters the untreated underlying portion, and so can become somewhat spread out in the underlying portion. Thus, by controlling the depth of the coagulation zone, it is possible to control the depth at which the subsequent optical energy treatment is delivered, as well as the diameter of the region in which the subsequent optical energy treatment is delivered.

Treating tissue in a manner so as to create coagulation zones with reduced optical scattering is particularly advantageous for treating tissue with an overlying portion and an underlying portion having different make-ups and/or optical properties, such as, for example, the skin, which has epidermis overlying dermis, or the skin and subcutis, which has epidermis and dermis overlying subcutaneous fat. Any differences between one or more overlying portions and one or more underlying portions can be exploited in order to optimize the method and create optimal coagulation zones in the overlying portion to serve as beam paths in order to effectively treat the underlying portion. For example, in skin and subcutaneous tissue, the epidermis and dermis have higher water contents than underlying subcutaneous fat. This difference in water content between the overlying and underlying portions can be beneficially used to produce optical energy treatments which have different effects on the epidermis and dermis than on the subcutaneous fat. For example, a wavelength of optical energy that is primarily absorbed by water can be used for the first optical energy treatment in order to create coagulation zones in the epidermis and dermis, and a wavelength that is primarily absorbed by fat can be used to for the subsequent optical energy treatment in order to melt or ablate subcutaneous fat. Differences in levels of chromophores or pigmentation can be similarly used to optimize the first and subsequent treatment parameters. Thus, a first treatment that is optimized to coagulate the overlying epidermis but not the underlying dermis can be applied so as to create coagulated zones in the epidermis, and a subsequent treatment that is optimized to effectively treat a condition present in the underlying dermis can then be aimed down the beam path created by the coagulation zone in the epidermis and into the dermis.

Various spot sizes can be used to deliver the first and subsequent optical energy treatments, depending upon the purpose of the treatment and the desired treatment effects. For example, the spot size of the first optical energy treatment can be in the range between about 30 μm and about 2 mm. In another example, the spot size of the first optical energy treatment can be in the range between about 50 μm and about 100 μm. In another example, the spot size of the first optical energy treatment can be in the range between about 100 μm and about 500 μm. In one example, the spot size of the subsequent optical energy treatments can be in the range between about 30 μm and about 2 mm. In another example, the spot size of the subsequent optical energy treatments can be in the range between about 50 μm and about 100 μm. In another example, the spot size of the subsequent optical energy treatments can be in the range between about 100 μm and about 500 μm.

The relative spot sizes for the first and subsequent optical energy treatments can be selected in order to optimize coupling of the treatments. For example, a larger spot size can be used to deliver the first optical energy treatment to create a larger coagulation zone and a smaller spot size can then be used to deliver the subsequent optical energy treatment in order to facilitate aiming the subsequent treatment beam down the coagulated beam path created by the first treatment.

Alternatively, a small spot size can be used to create a plurality of small coagulation zones using the first optical energy treatment, and a larger spot size can be used for the subsequent optical energy treatment, wherein the spot size of the subsequent optical energy treatment is large enough to illuminate more than one of the coagulated zones created by the first optical energy treatment. This avoids the need to accurately align the subsequent optical energy treatment with the coagulated zones created by the first optical energy treatment.

In another example, coagulated zones can be created using a first optical energy treatment which not only facilitates delivery of a subsequent optical energy treatment, but also facilitates detection of subsurface targets by a detector. Based on feedback from a detector that is configured to detect subsurface targets, the subsequent optical energy treatment can be delivered through the coagulated zones to a subsurface target detected by the detector. In another example, feedback from a detector that is configured to detect subsurface targets present below the coagulated zones can be used by the controller so as to deliver the subsequent optical energy treatment only through the coagulated zones where the desired subsurface target was detected.

The wavelength of the optical energy used for the first and subsequent optical energy treatments can be between about 1,200 nm and about 20,000 nm. The wavelength of the optical energy can be selected based on the absorption strength of various components within the tissue and the scattering strength of the tissue. The wavelength of the first and subsequent treatment can be chosen to target a particular chromophore, such as, for example, water, elastin, collagen, sebum, hemoglobin, myoglobin, melanin, keratin, or other molecules present in the tissue. Wavelengths that are primarily absorbed by water present in the tissue, such as, for example, 1550 nm can be used. Histological data and confocal microscopy studies have shown that thermally coagulated tissue is highly transparent in the near infrared spectrum and exhibits reduced light scattering properties, the wavelength of the subsequent optical energy treatment can be within the near infrared spectrum, such as, for example between about 700 nm and about 1400 nm. Wavelengths in the visible spectrum, such as, for example, between about 400 nm and about 700 nm are also useful, as the near infrared and visible spectrums typically are significantly scattered by untreated tissue. Ultra violet radiation within the range of between about 200 nm to about 400 nm can be used for the subsequent optical energy treatment as it is particularly effective for allowing lower levels of radiation to be used to activate photodynamic therapeutic agents for treatment of conditions in the papillary and reticular dermis.

Depending on the desired size and depth of the coagulation zones and treatment zones, the wavelength of the optical energy used can be selected from the group consisting of between about 1100 nm and about 2500 nm, between about 1280 nm and about 1350 nm, between about 1400 nm and about 1500 nm, between about 1500 nm and about 1620 nm, between about 1780 nm and 2000 nm, and combinations thereof. Wavelengths longer than 1500 nm and wavelengths with absorption coefficients in water of between about 1 cm⁻¹ and about 30 cm⁻¹ can be used if the goal is to get deep penetration with small coagulation zones or treatment zones. The shorter wavelengths generally have higher scattering coefficients than the longer wavelengths.

In one example, the same approximate wavelength of optical energy can be used for the first and subsequent optical energy treatments. In another example, different wavelengths of optical energy can be used for the first and subsequent optical energy treatments. In yet another example, the subsequent treatment can consist of more than one optical energy treatments each having different wavelengths. The choice of which wavelength or wavelengths to use for the first and subsequent treatments can be optimized based on factors, such as, for example, the desired diameter and depth of the coagulated zone, the desired diameter and depth of the treatment zone, the type(s) of tissue being treated, the type(s) of condition(s) being treated, the light scattering properties of the wavelength(s), etc.

Various forms of optical energy can be used in accordance with the methods and devices disclosed herein. The electromagnetic radiation can be coherent in nature, such as laser radiation, or non-coherent in nature, such as flashlamp radiation. Coherent electromagnetic radiation can be produced by lasers, including gas lasers, dye lasers, metal-vapor lasers, fiber lasers, diode lasers, and/or solid-state lasers. The type of laser used with this invention can be selected from the group consisting of an argon ion gas laser, a carbon dioxide (CO2) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, an erbium doped fiber laser, a neodymium doped fiber laser, a thulium doped fiber laser, an erbium-ytterbium co-doped fiber laser, and combinations thereof The laser can be applied in a fractional manner to produce fractional treatment. For example, the FRAXEL® SR 1500 laser (Reliant Technologies, Inc. Mountain View, Calif.) produces fractional treatment using an erbium-doped fiber laser operating at a wavelength that is primarily absorbed by water in tissue, at about 1550 nm.

The detector can be configured to detect a subsurface target either based on looking at the overlying portion of tissue or at the coagulation zones created in the overlying portion of tissue by the first optical energy treatment. The detector can be configured to detect a subsurface target based on detecting the presence of a particular chromophore. Alternatively, the detector can detect a subsurface target based on temperature, energy diffraction, energy absorption, energy scattering, surface reflection, particular wavelengths of energy, capacitance, etc. In one example, the detector can detect a form of electromagnetic energy. In another example, the detector can be a charge-coupled device, such as, for example, a silicon charge-coupled detector array. In another example, the detector can be a commercially available infrared camera. In yet another example, the detector can be a commercially available near-infrared camera capable of detecting optical energy of wavelengths between about 700 nanometers and about 1000 nanometers. In another example, the detector can use the effect of a form of diagnostic energy on the tissue to detect the presence of a particular molecule such as, for example, water, hemoglobin, melanin, myoglobin, lipids, sebum, phytosphingosine, etc, found in or near skin, hair, follicles and/or their surrounding tissue. In another example, the detector can use the effect of a form of diagnostic energy on the tissue to detect the presence of a particular structure below the level of the skin, such as, for example, the opening of a follicle at the surface of the skin, all or a portion of a follicle below the surface of the skin, a sebaceous gland, a hair bulge, a hair bulb, a capillary structure surrounding a follicle, etc. Similarly, the feedback generated by the detector can be of various forms, such as of thermal data, thermal images, infrared data, infrared images, diffraction patterns, absorption spectra, levels of scattering of optical energy, the presence or absence of colors, capacitance data, etc.

The process by which the detector detects the presence of a subsurface target can be completely automated. Alternatively, the process by which the detector detects the presence of subsurface target can rely in part on input from an operator. In one example, the detector can consist of one detector that detects the presence of a hair and/or follicle. In another example, the detector can consist of multiple detectors that detect a hair and/or follicle.

While these methods and devices can be used for medical and/or cosmetic or purposes to remodel tissue (for example, for collagen remodeling), to resurface tissue, to treat wrinkles and photoaging of the skin, to remove pigmented lesions, to remove tattoos, and/or to remove hair, they are also suitable to treat a variety of dermatological condition such as hypervascular lesions including port wine stains, capillary hemangiomas, cherry angiomas, venous lakes, poikiloderma of civate, angiokeratomas, spider angiomas, facial telangiectasias, telangiectatic leg veins; pigmented lesions including lentigines, ephelides, nevus of Ito, nevus of Ota, Hori's macules, keratoses pilaris; acne scars, epidermal nevus, Bowen's disease, actinic keratoses, actinic cheilitis, oral florid papillomatosis, seborrheic keratoses, syringomas, trichoepitheliomas, trichilemmomas, xanthelasma, apocrine hidrocystoma, verruca, adenoma sebacum, angiokeratomas, angiolymphoid hyperplasia, pearly penile papules, venous lakes, rosacea, etc. While specific examples of dermatological conditions are mentioned above, it is contemplated that these methods and devices can be used to treat virtually any type of dermatological condition.

Additionally, these methods and devices can be applied to other medical specialties besides dermatology. The inventions disclosed herein are also applicable to treatment of other tissues of the body. For example, the treatment of heart tissue can also benefit from the use of this invention.

Although the detailed description contains many specifics, these should not be construed as limiting the scope of the invention but merely as illustrating different examples and aspects of the invention. It should be appreciated that the scope of the invention includes other embodiments not discussed in detail above. For example, the inventions disclosed herein can be generalized to RF, flashlamp, or other electromagnetic energy based treatments as well. Various other modifications, changes and variations which will be apparent to those skilled in the art may be made in the arrangement, operation and details of the methods and devices of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

All publications, patents and patent applications cited herein, whether supra or infra, are hereby incorporated by reference in their entirety.

In the specification and in the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

### EXAMPLES

### Example 1

### Investigation of Treatment Depth through a Coagulation Zone with First and Subsequent Treatments Using the Same Wavelength

The influence and benefit of treating through coagulated beam paths are investigated using an experimental set-up involving initially a treatment using a single coagulative wavelength. A 1550 nm laser is used to perform time-resolved measurements on ex vivo human tissue. Two consecutive laser pulses at a wavelength of 1550 nm are delivered with a temporal separation long enough (1-2 minutes) for the treated tissue to return back to its baseline temperature before the subsequent pulse is delivered.

Tissue is irradiated repeatedly using an investigational galvanometer-based scanning mechanism combined with a 1550 nm fiber laser (IPG Photonics, Oxford, Mass., USA). The galvanometer-based scanning mechanism is controlled using WaveRunner Laser/Scanner Software (Nutfield Technology, Inc., Windham, N.H., USA). Initially, a laser spot size of 1 mm or larger is used with a pulse energy of 500 to 600 mJ corresponding to an incident fluence of 64 to 76 J/cm². The goal of this first treatment is to coagulate tissue without causing mechanical surface disruption or disruption of the dermal-epidermal junction. If necessary, the beam profile is flattened in order to avoid the central hot spot found on true Gaussian beam profiles. Evaporation of water from the tissue between the first and subsequent treatments is minimized by occluding the skin surface with a thin film of transparent plastic or a microscope cover slip. Once the tissue is allowed to cool following the first treatment, one or more subsequent treatments are delivered to the same location in the tissue. The one or more subsequent pulse(s) are of equal, greater, or lesser energy as compared to the first pulse. Histological analysis using hematoxylin and eosin (H&E) stain as well as observation of the tissue under cross polarized light is used to confirm the depth of tissue coagulation following the first and subsequent pulses.

As there will not be a significant change in the water content of the tissue during the investigation, and the tissue is permitted to cool down between laser pulses, and the subsequent laser pulse(s) delivered to the same location will cause heating of tissue in a manner similar to the first pulse. However, since the tissue is coagulated by the first treatment, the subsequent pulse(s) delivered to the same region of tissue exhibit reduced light scattering. Consequently, the subsequent laser pulse(s) have a deeper penetration depth into the tissue, and, as the subsequent pulse(s) will be coagulative in nature, the depth of the coagulation zone in the treated region of tissue increase as the one or more subsequent pulses are applied to the tissue. By varying the energy of the subsequent pulse(s), the optimal energy required for the subsequent treatment to reach a desired tissue depth is determined.

### Example 2

### Treatment of Cellulite and/or Subcutaneous Fat

Cellulite and/or subcutaneous fat is treated using the apparatus described in Example 1 with the addition of a second optical energy source with a wavelength that is absorbed by fat. Examples of wavelengths significantly absorbed by fat include, but are not limited to, about 915 nm to about 920 nm, about 1210 nm, and about 1720 nm. In this Example, the laser described in Example 1 is used to provide the first treatment, while a second optical energy source with a wavelength that is absorbed by fat is used to provide the one or more subsequent treatments. The second optical energy source is configured in a manner such that its beam is coaxial with respect to the first coagulating laser beam, which in this example is a 1550 nm fiber laser.

In the investigative portion of this Example, ex vivo tissue is treated first with the first coagulative treatment at 1550 nm and allowed to return to its baseline temperature. The one or more subsequent treatments at the wavelength absorbed by fat are then be administered to coagulation zones created by the first treatment. A pulse energy of about 500 mJ to about 1000 mJ is used for the one or more subsequent treatments. The tissue response is evaluated histologically as described in Example 1 in order to evaluate treatment effects.

Additionally, the effects of variable temporal delays between the first coagulative treatment and the subsequent one or more fat-specific laser wavelength treatments are determined using a variable time delay pulse generator. The delay time between the first and subsequent pulses is varied between about 100 ms and about 2 seconds. The tissue response is evaluated histologically as described in Example 1 in order to evaluate treatment effects.

The tissue responses to the various treatments tested in the first portion of Example 2 is evaluated, and the optimal treatment parameters is used to treat human volunteers in order to reduce subcutaneous fat and/or reduce the appearance of cellulite.

### Example 3

### Treatment of Sebaceous Glands

The apparatus of Example 2, with the addition of a detector configured to detect sebaceous glands and/or follicles at the surface of the skin, is used to treat sebaceous glands in order to reduce their ability to produce sebum. An investigative study in ex vivo tissue is conducted as in Example 2 in order to determine optimal treatment parameters such as, for example, pulse energy and temporal delay between the first and subsequent treatments, with tissue responses evaluated histologically as described in Example 1. Once the optimal treatment parameters are determined, these parameters are used to treat human volunteers in order to reduce sebum production.

### Example 4

### Treatment of Hair

The apparatus of Example 2, with the addition of a detector configured to hairs on the surface of the skin and/or follicles, are used to treat the hair bulge and/or hair bulb in order to reduce or delay the ability of a hair follice to regenerate a hair. An investigative study in ex vivo tissue is conducted as in Example 2 in order to determine optimal treatment parameters such as, for example, pulse energy and temporal delay between the first and subsequent treatments, with tissue responses evaluated histologically as described in Example 1. Once the optimal treatment parameters are determined, these parameters are used to treat human volunteers in order to remove hair and/or delay hair growth.

### Example 5

### Treatment of Vascular Lesions

Treatment of deep subsurface targets can include vascular lesions. Wavelengths between about 400 nm and about 1064 nm are well absorbed by the chromophores present in blood, such as, for example, various forms of hemoglobin, and are used to treat vascular lesions. For example, green laser light with a wavelength of about 532 nm is used to treat vascular targets. In order to examine the dynamics of reduced light scattering within coagulated beam paths, an EPIX high-speed camera and software (EPIX, Inc., Buffalo Grove, Ill., USA) with capabilities of recording up to 1000 frames per second are set up to record the transmission of a green laser through a 100 micron thick dermal and epidermal tissue section mounted onto a microscope slide. The visible laser and a near infrared laser such as a 1550 nm fiber laser (as described in Example 1) with beam sizes of 1 mm is coaxially aligned as described in Example 2. The camera uses a near infra-red (NIR) cut-off filter to eliminate any 1550 nm light from being recorded by the camera.

The first (1550 nm) treatment and the one or more subsequent (532 nm) treatments are applied to ex vivo tissue. The visible light transmitted through the tissue sample is either imaged directly onto the camera or the reflection from a screen is recorded during the first 1550 nm laser pulse which coagulates the tissue. Changes in the transmitted intensity as well as beam shape are related directly to changes in the tissue. Again, evaporation of surface water is minimized by keeping the tissue covered with a microscope slide. The tissue response to the first and subsequent treatments is evaluated histologically as described in Example 1. As the ex vivo human skin samples are microtomed, the epidermis and dermis is evaluated separately, as can defatted full thickness skin.

The optimal parameters for the first 1550 nm laser treatment are varied and evaluated in order to produce optimal coagulated beam paths while avoiding mechanical disruption of the stratum corneum as well as the dermal-epidermal junction. The optimal parameters for the subsequent 532 nm laser treatment are also be varied as well and evaluated in order to produce the optimal treatment of the vascular lesion. The optimal first and subsequent treatment parameters are used to treat human volunteers in order to remove vascular lesions.

What is claimed is:

1. A method of treating tissue having an overlying portion and an underlying portion, the method comprising:
treating an overlying portion of tissue with a first optical energy treatment in a fractional manner so as to thermally coagulate a plurality of fractions in the overlying portion of a region of tissue, thereby creating a plurality of coagulated zones having reduced light scattering as compared to equivalent sized zones of untreated overlying tissue; and
treating an underlying portion of tissue with a subsequent optical energy treatment in a fractional manner so as to effectively treat a condition present in the underlying portion of tissue, thereby creating at least one treatment zone in the underlying portion of tissue, wherein the subsequent optical energy treatment is directed through at least one of the plurality of coagulated zones in the overlying portion to form the at least one treatment zone in the underlying portion of a region of tissue.

2. The method of claim 1, wherein the method further comprises detecting a subsurface target in the region of tissue.

3. The method of claim 1, wherein the method further comprises detecting a subsurface target in the region of tissue by detecting through the coagulated zone.

4. The method of claim 1, wherein the treating an underlying portion of tissue comprises delivering the subsequent optical energy treatment to a detected subsurface target.

5. The method of claim 1, wherein the treating an underlying portion of tissue comprises delivering the subsequent optical energy treatment to a subset of the plurality of coagulated zones, wherein the subset of coagulated zones comprise coagulated zones in which a subsurface target was detected.

6. The method of claim 1, wherein the first optical energy treatment comprises one optical energy treatment.

7. The method of claim 1, wherein the first optical energy treatment comprises more than one optical energy treatment.

8. The method of claim 1, wherein the first optical energy treatment ablates a portion of the overlying portion and coagulates a portion of the overlying portion.

9. The method of claim 1, wherein the subsequent optical energy treatment comprises one optical energy treatment.

10. The method of claim 1, wherein the subsequent optical energy treatment comprises more than one optical energy treatment.

11. The method of claim 1, wherein the wavelength of both the first optical energy treatment and the subsequent optical energy treatment is between about 1,200 nm and about 20,000 nm.

12. The method of claim 1, wherein the wavelength of both the first optical energy treatment and the second optical energy treatment is strongly absorbed by water.

13. The method of claim 1, wherein the wavelength of the first optical energy treatment is in the near infrared spectrum.

14. The method of claim 1, wherein the wavelength of the first optical energy treatment is between about 700 nm and about 1400 nm.

15. The method of claim 1, wherein the first optical energy treatment is produced by a laser selected from the group consisting of an argon ion gas laser, a carbon dioxide (CO2) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, an erbium doped fiber laser, a neodymium doped fiber laser, a thulium doped fiber laser, an erbium-ytterbium co-doped fiber laser, and combinations thereof.

16. The method of claim 1, wherein the subsequent optical energy treatment is produced by a laser selected from the group consisting of an argon ion gas laser, a carbon dioxide (CO2) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, an erbium doped fiber laser, a neodymium doped fiber laser, a thulium doped fiber laser, an erbium-ytterbium co-doped fiber laser, and combinations thereof.

17. The method of claim 1, wherein the first and subsequent optical energy treatments have the same wavelength.

18. The method of claim 1, wherein the first and subsequent optical energy treatments have different wavelengths.

19. The method of claim 1, wherein the subsequent optical energy treatment immediately follows the first optical energy treatment.

20. The method of claim 1, wherein the subsequent optical energy treatment overlaps in time with the first optical energy treatment.

21. The method of claim 1, wherein there is a gap in time between the first optical energy treatment and the subsequent optical energy treatment.

22. The method of claim 1, wherein the spot size of the first optical energy treatment is between about 30 μm and about 2 mm.

23. The method of claim 1, wherein the spot size of the first optical energy treatment is between about 50 μm and about 1000 μm.

24. The method of claim 1, wherein the spot size of the first optical energy treatment is between about 100 μm and about 500 μm.

25. The method of claim 1, wherein the spot size of the subsequent optical energy treatment is between about 30 μm and about 2 mm.

26. The method of claim 1, wherein the spot size of the subsequent optical energy treatment is between about 50 μm and about 1000 μm.

27. The method of claim 1, wherein the spot size of the subsequent optical energy treatment is between about 100 μm and about 500 μm.

28. The method of claim 1, wherein the spot size of the first optical energy treatment is larger than the spot size of the subsequent optical energy treatment.

29. The method of claim 1, wherein the spot size of the first optical energy treatment is smaller than the spot size of the subsequent optical energy treatment.

30. The method of claim 1, wherein the spot size of the first and subsequent optical energy treatment are approximately equal.

31. The method of claim 1, wherein the overlying portion is epidermis and the underlying portion is dermis.

32. The method of claim 1, wherein the overlying portion is skin, and the underlying portion is subcutis.

33. The method of claim 1, wherein the wavelength of the subsequent optical energy treatment is a wavelength that is absorbed by fat, and the subsequent optical energy treatment is directed to a region of subcutaneous fat in order to reduce the volume of subcutaneous fat.

34. The method of claim 1, wherein the wavelength of the subsequent optical energy treatment is a wavelength that is absorbed by fat, and the subsequent optical energy treatment is directed to a region of cellulite in order to reduce the appearance of cellulite.

35. The method of claim 1, wherein the wavelength of the subsequent optical energy treatment is a wavelength that is absorbed by a form of hemoglobin, and the subsequent optical energy treatment is directed to a vascular lesion in order to remove the vascular lesion.

36. The method of claim 1, wherein the wavelength of the subsequent optical energy treatment is a wavelength that is absorbed by a tattoo ink, and the subsequent optical energy treatment is directed to a region of tissue containing a tattoo in order to remove the tattoo.

37. A device for providing an optical energy treatment to a region of tissue having an overlying portion and an underlying portion, comprising:
an optical energy source for providing a first optical energy treatment configured to apply the first optical energy treatment in a fractional manner so as to thermally coagulate a plurality of fractions of a overlying portion of a region of tissue, thereby creating a plurality of coagulated zones having reduced light scattering as compared to equivalent sized zones of untreated overlying tissue;
an optical energy source for providing a subsequent optical energy treatment configured to apply the subsequent optical energy treatment in a fractional manner so as to effectively treat a condition present in the underlying portion of tissue, thereby creating at least one treatment zone in the underlying portion of tissue, wherein the subsequent optical energy treatment is directed through at least one of the plurality of coagulated zones in the overlying layer to form the at least one treatment zone in the underlying portion of the region of tissue;
a controller configured to control the optical energy source or sources providing the first and subsequent optical energy treatments; and
a detector configured to detect the presence of a subsurface target through the plurality of coagulated zones and to provide feedback to the controller;
wherein the controller uses the feedback from the detector to determine whether or not to apply the subsequent optical energy treatment to a detected subsurface target through the at least one of the plurality of coagulated zones in order to effectively treat the detected subsurface target by creating the at least one treatment zone in the underlying portion.

38. The device of claim 37, wherein the optical energy sources for providing the first and subsequent optical energy treatments have the same wavelength.

39. The device of claim 37, wherein the optical energy sources for providing the first and subsequent optical energy treatments have different wavelengths.

40. The device of claim 37, wherein the subsequent optical energy treatment is delivered immediately following the first optical energy treatment.

41. The device of claim 37, wherein the subsequent optical energy treatment is delivered in a manner so as to overlap in time with the first optical energy treatment.

42. The device of claim 37, wherein there is a gap in time between the delivery of the first optical energy treatment and the delivery of the subsequent optical energy treatment.

43. The device of claim 37, wherein the spot size of the first optical energy treatment is larger than the spot size of the subsequent optical energy treatment.

44. The device of claim 37, wherein the spot size of the first optical energy treatment is smaller than the spot size of the subsequent optical energy treatment.

45. The device of claim 37, wherein the spot size of the first and subsequent optical energy treatments are approximately equal.

46. The device of claim 37, wherein the spot size of the first optical energy treatment is between about 30 μm and about 2 mm.

47. The device of claim 37, wherein the spot size of the first optical energy treatment is between about 50 μm and about 1000 μm.

48. The device of claim 37, wherein the spot size of the first optical energy treatment is between about 100 μm and about 500 μm.

49. The device of claim 37, wherein the spot size of the subsequent optical energy treatment is between about 30 μm and about 2 mm.

50. The device of claim 37, wherein the spot size of the subsequent optical energy treatment is between about 50 μm and about 1000 μm.

51. The device of claim 37, wherein the spot size of the subsequent optical energy treatment is between about 100 μm and about 500 μm.

52. The device of claim 37, wherein the spot size of the first optical energy treatment is larger than the spot size of the subsequent optical energy treatment.

53. The device of claim 37, wherein the detector comprises a detector that detects a form of electromagnetic energy, diffraction, absorption, electromagnetic energy scatter, color, capacitance, the presence of water, the presence of sebum, the presence of melanin, the presence of a hair, the presence of a follicle, of the presence of a vasculature structure.
